(19) United States
(12) Patent Application Publication
(10) Pub. No.: US 2005/0171192 A1
(43) Pub. Date: Aug. 4, 2005

(54) Use of histamine to treat bone disease

(76) Inventor: Kurt R. Gehlsen, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/007,905

(22) Filed: Dec. 8, 2004

Related U.S. Application Data
(60) Provisional application No. 60/529,205, filed on Dec. 11, 2003.

(51) Int. Cl. A61K 38/44; A61K 31/555; A61K 31/355

(52) U.S. Cl. 514/458; 514/474; 514/725; 424/94.4; 514/400

(57) ABSTRACT

Described herein are methods for treating and/or preventing bone tissue and cell damage caused by reactive oxygen species in mammals. More specifically, embodiments of the invention relate to the prevention and/or reduction of bone tissue and cell damage through the administration of histamine, histamine agonists, and related compounds.

### RELATED APPLICATIONS

This application claims priority under 35 U.S.C. § 119(e) to U.S. Provisional Application Ser. No. 60/529,205, filed on Dec. 11, 2003, which is hereby expressly incorporated by reference in its entirety.

### BACKGROUND OF THE INVENTION

1. Field of the Invention

Embodiments of the invention described herein relate to methods for treating and/or preventing bone tissue and cell damage caused by reactive oxygen species in mammals. More specifically, the disclosure relates to the prevention and/or reduction and/or reversal of bone tissue and cell damage through the administration of histamine and histamine-related compounds.

2. Description of the Related Art

Bones are living, growing tissues that are in a constant state of change, with old tissue being broken down (resorption) and new tissue formed in its place (formation). The fine balance between bone resorption and bone formation is maintained by osteoclast cells which continuously break down or demineralize old tissue and aid in the shaping of new growth, and osteoblast cells which continuously form new tissue for growth or repair of damage to the bone.

Osteoclasts are multinuclear, haematopoietic cells of the monocyte and macrophage lineage. Osteoclasts demineralize bones through extracellular bone dissolution, a process involving the secretion of hydrolytic enzymes and protons and the generation of reactive oxygen species (ROS). Berger et al., J. Endocrinology 158: 311-18 (1998).

Oxidative stress, i.e. toxicity inflicted by ROS, is being recognized as a systemic phenomenon in bone disease, whose extent appears to correlate with the severity and stage of disease. The mechanism of action associated with the cellular damage caused by oxidative stress has been implicated in a number of diseases and relates to direct damage of bone tissue. Examples of such diseases include osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone.

The theory that oxidative stress may play a role in bone disease may not be surprising as oxidative stress has been proposed to contribute to the state of immunosuppression at the site of malignant tumors and in chronic viral infections. (See U.S. Pat. Nos. 5,728,378, 6,000,516, and 6,155,266). Lymphocytes residing within or adjacent to tumors display signs of oxidative damage, including a higher degree of apoptosis and a defective transmembraneous signal transduction. The oxidative stress at the site of tumor growth is presumably conveyed by ROS produced by adjacent phagocytic cells (monocyte/macrophages (MO) or neutrophilic granulocytes (GR)). Histamine, an inhibitor of ROS production in phagocytes, is currently used as an adjunct to lymphocyte-activating cytokines (IL-2 and IFN-alpha) with the aim to enhance cytokine efficiency.

The complete reduction of one molecule of O₂ to water is a four-electron process. Oxidative metabolism continually generates partially reduced species of oxygen, which are far more reactive, and hence more toxic than O₂ itself. A one-electron reduction of O₂ yields superoxide ion (O₂⁻); reduction by an additional electron yields hydrogen peroxide (H₂O₂), and reduction by a third electron yields a hydroxyl radical (OH.), and a hydroxide ion. Nitrous oxide (NO), is another interesting reactive oxygen metabolite, produced through an alternative pathway. Hydroxyl radicals in particular are extremely reactive and represent the most active mutagen derived from ionizing radiation. All of these species are generated and must be converted to less reactive species if the organism is to survive.

Particular cells of the immune system have harnessed the toxic effects of ROS as an effector mechanism. Professional phagocytes, polymorphonuclear leukocytes (neutrophils, PMN), monocytes, macrophages, and eosinophils function to protect the host in which they reside from infection by seeking out and destroying invading microbes. Similarly, osteoclasts exploit the toxic effects of ROS to aid in bone resorption. These phagocytic cells possess a membrane-bound enzyme system which can be activated to produce toxic oxygen radicals in response to a wide variety of stimuli.

The “increased respiration of phagocytosis” (the respiratory burst) was reported and thought to be a result of increased mitochondrial activity providing additional energy for the processes of phagocytosis. It was later shown that a non-mitochondrial enzymatic system produced the increased levels of oxygen metabolites since the respiratory burst continued even in the presence of mitochondrial inhibitors such as cyanide and antimycin A. In 1968, Paul and Sbarra showed clearly that hydrogen peroxide was produced by stimulated phagocytes and in 1973 Babior and co-workers established that superoxide was a major product of the oxidase. (Paul and Sbarra, Biochim Biophys Acta 156(1): 168-78 (1968); Babior, et al., J. Clin Invest 52(3): 741-4 (1973). It is now generally accepted that the enzyme is membrane bound, exhibits a preference for NADPH (Kₘ=45 μM) over NADH (Kₘ=450 μM, and converts oxygen to its one electron-reduced product, superoxide.
NADPH+H⁺+2O_{2→NADP}⁺+2H⁺2O₂⁻
The hydrogen peroxide arises from subsequent dismutation of the superoxide.
2O₂⁻+2H⁺→H₂O₂+O₂⁻

While there are beneficial effects of these oxygen metabolites, it is clear that inappropriate production of oxygen metabolites can result in severely deleterious effects. Several disease states illustrate this point, including various bone diseases, such as osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone. An effective method to reduce and/or prevent the production and release of ROS in patients suffering from or at risk for bone disease would be a great boon to medicine and serve to reduce and eliminate a substantial amount of human suffering.

Given the ravaging effects of bone disease and the only partially successful treatment methods available today, there is a constant demand for improved methods of treating bone disease and reducing bone cell death and bone loss.

### SUMMARY OF THE INVENTION

Embodiments of the invention relate to methods for treating and/or preventing tissue and cell damage caused by reactive oxygen species (ROS) in mammals. More specifically, embodiments of the invention relate to the prevention and/or reduction of bone tissue and bone cell damage through the administration of histamine and histamine agonists.

In some embodiments, the invention described herein relates to methods for treating and/or preventing bone tissue and cell damage caused by reactive oxygen species in mammals. More specifically, the disclosure relates to the prevention of bone cell death and bone resorption through the administration of histamine and related compounds. In other embodiments, the invention relates to methods for reducing or preventing cell death or apoptosis in bone cells. Bone cells include, for example, osteoclasts, osteoblasts, and osteocytes. In one embodiment, a method for treating or preventing reactive oxygen species (ROS)-mediated oxidative damage to bone cells and tissues of a subject is provided, comprising the step of administering a compound that reduces the amount of ROS to a subject suffering from or at risk for a bone disease caused or exacerbated by ROS-mediated oxidative damage. In some embodiments the ROS-mediated damage is enzymatically produced damage. In alternative embodiments, the ROS-mediated damage is cellular derived, such as osteoclast-derived damage. In some embodiments, the amount of ROS is reduced by inhibiting the production or release of ROS. Although the compositions and methods are applicable to any bone disease, they are particularly relevant to the treatment of bone diseases selected from the group consisting of osteoporosis, including, but not limited to, type I and type II osteoporosis, age-related osteoporosis, disuse osteoporosis, diabetes-related osteoporosis, and steroid-related osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone.

Another embodiment relates to a method for treating a subject suffering from a disease state wherein osteoclast-produced, reactive oxygen species (ROS)-mediated oxidative damage can occur, which comprises identifying a subject with a bone disease in which ROS cause ROS-meditated oxidative damage and administering a compound effective to reduce the amount of ROS.

Subjects suffering from or at risk for bone loss can be identified by methods known in the art, such as, for example, by radiographic measurement of bone density, by evaluation of biochemical markers such as alkaline phosphatase, osteocalcin, urinary calcium, and urinary hydroxyproline, by bone biopsy with pathological assessment, and by assessment of family history. Examples of bone density techniques include, for example, single- and dual photon absorptiometry, quantitative computed tomography, dual x-ray absorptiometry, and ultrasonography. Preferred sites of analysis include the hip, wrist, and vertebrae. Other detection methods include low level x-ray on a finger or wrist, ultrasound of the heel, and CT scan of the spine.

Advantageously, the compound effective to reduce the amount of ROS is a compound that inhibits the production or release of cellular-derived or enzymatically released reactive oxygen species. In some embodiments, the compound effective to inhibit the production or release of ROS is histamine, a histamine receptor agonist, a NADPH oxidase inhibitor, serotonin or a serotonin agonist. Optionally, the composition further includes an effective amount of a ROS scavenger. The ROS scavenger can be catalase, superoxide dismutase, glutathione peroxidase, or ascorbate peroxidase.

Optionally, the method further includes the step of administering an effective amount of a ROS scavenger. Advantageously, the step of administering said ROS scavenger results in ROS scavenger catalyzed decomposition of ROS. Such scavengers include catalase, superoxide dismutase, glutathione peroxidase, or ascorbate peroxidase. Additionally, the scavenger can be vitamin A, vitamin E, or vitamin C.

In still another embodiment of the invention, a method of reducing bone tissue damage associated with steroid and hormone treatment is provided. The method includes administering to a subject in need thereof an effective amount of a compound effective to inhibit the production or release of cellular-derived or enzymatically produced ROS. Advantageously, the compound to inhibit the production or release of ROS includes histamine, histamine receptor agonists, NADPH oxidase inhibitors, serotonin and serotonin agonists. Optionally, the method can include a further step of administering an effective amount of a ROS scavenger. Preferably, the step of administering the ROS scavenger results in ROS scavenger catalyzed decomposition of ROS. The scavenger can be catalase, glutathione peroxidase, superoxide dismutase, or ascorbate peroxidase, for example. Additionally, the scavenger can be vitamin A, vitamin E, or vitamin C.

### DETAILED DESCRIPTION

The disclosure below relates to compositions and methods for preventing and reducing bone cellular and tissue damage caused by reactive oxygen species (ROS).

Bones play an essential role in support, protection of internal organs from mechanical damage, as a reservoir of minerals such as calcium and phosphate, and as a source of all blood cells. Diseases of the bone typically have serious consequences for the person afflicted, ranging from morbidity to mortality. Examples of bone diseases include: osteoporosis, including, but not limited to, type I and type II osteoporosis, age-related osteoporosis, disuse osteoporosis, diabetes-related osteoporosis, and steroid-related osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone.

Recent work has indicated that these and other bone diseases may be exacerbated by ROS. ROS can have direct effects on various cells within the bones leading to apoptosis. Another possible mechanism by which these molecules can damage bone cells and tissue may be related to the role of ROS in bone resorption. For example, ROS produced by osteoclasts may effectively suppress bone formation and bone healing.

One embodiment of the invention relates to compositions and methods for treating and/or preventing cellular and tissue damage caused by reactive oxygen species released by osteoclasts in the process of bone resorption. In some embodiments, the compositions and methods of the invention reduce ROS-mediated damage by inhibiting the production or release of ROS.

A variety of reactive oxygen metabolites (ROMs) are produced in the monovalent pathway of oxygen reduction. These ROMs are enzymatically produced by osteoclasts and phagocytes such as monocytes and polymorphonuclear neutrophils (PMNs) and frequently released in a respiratory burst. Neutrophils also produce ROMs constitutively. The constitutive production may contribute to ROS-mediated cellular damage. Hydrogen peroxide and other ROS play an important role in a host's immunological defenses. Nevertheless, ROS produced in excessive amounts or at inappropriate times or locations, act to damage a host's cells and tissues, and thus can be detrimental to the host.

The effects of ROS production are many faceted. ROS are known to cause apoptosis in NK cells. ROS are also known to cause anergy and/or apoptosis in T-cells. The mechanisms by which ROS cause these effects are not yet fully understood. Nevertheless, some commentators believe that ROS cause cell death by disrupting cellular membranes and by changing the pH of cellular pathways critical for cell survival and also by direct damaging effects on DNA.

It is one of the surprising discoveries of the invention that compounds that reduce the amount of ROS produced or released by sources within a subject can facilitate the treatment and recovery of individuals suffering from bone loss. The conditions contemplated as treatable under the embodiments of the invention result from a disparate number of etiological causes. Nevertheless, they share a common feature in that their pathological conditions are either caused or exacerbated by enzymatically produced, ROS-mediated oxidative damage, caused by inappropriate and harmful concentrations of ROS. Thus, the administration of compounds that inhibit the production or release of ROS, or scavenge ROS, alone or in combination with other beneficial compounds, provides an effective treatment for a variety of bone diseases.

Embodiments of the invention contemplate compounds and methods that are efficacious in treating or preventing a variety of bone loss conditions wherein ROS play an active, detrimental role in the pathological state of the disease. Such conditions include but are not limited to: osteoporosis, including, but not limited to, type I and type II osteoporosis, age-related osteoporosis, disuse osteoporosis, diabetes-related osteoporosis, and steroid-related osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone.

The compounds which reduce the amount of ROS produced and released in an individual and the methods disclosed below are directed to the reduction and prevention of ROS-mediated damage of bone cells and tissue. In preferred embodiments, various histamine and histamine-related compounds are used to achieve a beneficial reduction or inhibition of enzymatic ROS production and release or the net concentration thereof. Histamine and histamine-related compounds include, for example, histamine, the dihydrochloride salt form of histamine (histamine dihydrochloride), histamine diphosphate, other histamine salts, histamine esters, histamine prodrugs, and histamine receptor agonists are to be included. Other ROS production and release inhibitory compounds such as NADPH oxidase inhibitors like diphenyleneiodonium can also be used with the disclosed methods, as can serotonin and 5HT-receptor agonists.

The administration of compounds that induce the release of endogenous histamine from a patient's own tissue stores is also included within the scope of the present disclosure. Such compounds include IL-3, retinoids, and allergens.

The compositions and methods disclosed herein also encompass the administration of a variety of ROS scavengers. Known scavengers of ROS include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROS-mediated damage. The scope of the methods disclosed herein includes the administration of the compounds listed and those compounds with similar ROS inhibitor activity. The compositions and methods disclosed herein also provide an effective means for preventing and/or inhibiting the release of enzymatically generated ROS in excessive amounts or at inappropriate times or locations.

Compounds and methods for treating bone disease states that are complicated by the detrimental release of ROS within a host or subject are provided. Bones are responsible for many essential functions in the body. The impairment of these functions by bone disease can lead to very serious consequences. Bone damage has been linked to a number of sources. They may be caused by infections with bacteria, or viruses.

Examples of environmental and industrial toxins which cause damage to bone tissue include, without limitation, cigarette smoke, caffeine, alcohol, detergents, petroleum products, radiation, diethanoloamine, sodium laurel sulfate, propylene glycol, pesticides such as DDT and mirex, food additives and preservatives, heavy metals, organic solvents such as formaldehyde and bromobenzene, and solvents such as dioxins, flurans, TCE, PCE, DCE, tetrachloroethylene, carbon tetrachloride, and vinyl chloride. As will be described in greater detail below, toxins also include many common drugs, such as steroids, chemotherapy drugs, hormones, and anticonvulsants. Damage to bone tissue results, at least in part, by the detrimental release of ROS within a host or subject in response to such insults. Accordingly, compositions and methods for treating damage to bone tissue caused by exposure to toxic substances are provided. Specifically, the administration of a ROS production and release inhibiting compound is useful for the reduction in trauma to bone cells and tissues following exposure to industrial and/or environmental toxins.

Numerous medications have been associated with damage to the bones. Such drugs include any substance or substances which act upon the bones to cause tissue damage. Examples of medications that have been associated with bone loss include, without limitation, corticosteroids, such as betamethasone, budesonide, cortisone dexamethasone, hydrocortisone, methylprednisolone, prednisolone, prednisone, and triamcinolone; cancer treatments, such as hormone therapy, including, for example, androgen deprivation in prostrate cancer, orchiectomy, hormone therapy such as reduced estrogen and/or progesterone for breast cancer or metastatic breast cancer; thyroid hormone, such as thyroxine, for hyperthyroidism; anticonvulsants, such as barbituates, phenoarbital, phenyloin, and benzodiazepines; and lupus and Crohn's disease treatments.

Accordingly, ROS inhibiting or scavenging compounds can be administered to an individual who is concurrently taking a drug or drugs which cause toxic side effects to mitigate bone loss caused by the drug. In one embodiment, an individual taking a drug associated with bone loss is administered an effective amount of a ROS inhibiting compound or scavenger separately or as a single formulation with the drug. The ROS inhibiting compound or scavenger and toxic drug can be given substantially simultaneously or within various time durations of each other. The administration can be by either local or by systemic injection or infusion. Other methods of administration may also be suitable, such as by oral route.

The administration of a ROS inhibitor or scavenger is likewise useful for ameliorating damage to bone tissue caused or exacerbated by bacterial, or viral infections. Staphylococcus aureus, Streptococcus pyogenes, Haemophilus influenzae, Myobacterium tuberculosis, salmonellas and coliform bacteria, Pseudomonas aeruginosa, Treponema pallidum, and Escherichia coli are just a few examples of a species of pathogenic bacteria which invades the bones and causes tissue damage.

Accordingly, in one embodiment, compounds and methods for minimizing damage to bone tissue associated with bacterial, fungal, or viral infections are provided. ROS production and release inhibiting compounds are administered alone or in combination with an antibiotic. As used herein, the term “antibiotic” includes any antibacterial, or antifungal compound. When administered in combination with antibiotics, the ROS production and release inhibiting compound can be administered separately or as a single formulation with the antibiotic. If administered separately, the ROS production and release inhibiting compound should be given in a temporally proximate manner such that the amelioration of damage to bone tissue is enhanced. In one embodiment, the ROS production and release inhibiting compound and antibiotic are given within one week of each other. In another embodiment, the ROS production and release inhibiting compound and antibiotic are given within twenty-four hours of each other. In yet another embodiment, the ROS production and release inhibiting compound and antibiotic are given within one hour of each other. The administration can be by either local or by systemic delivery. Other methods of administration may also be suitable, such as oral administration.

In yet another embodiment, compositions and methods for treating bone diseases secondary to other disease etiologies are provided. For example, anorexia, amenorrhea, and bulimia often leads to bone loss. Similarly, celiac disease (an intolerance of grain), diabetes, thyroid diseases such as hyperthyroidism and hypothyroidism, sickle cell anemia, asthma, gastrointestinal disorders such as blocked intestinal absorption of calcium due to chronic diarrhea, rheumatoid arthritis, lupus, hypercalciuria, and kidney or liver disease can also lead to bone loss. In addition, while primary cancer of the bone is rare, it is common for cancer to spread to the bones, such as the spine, skull, hip, pelvis, and long arm and leg bones, as a secondary metastatic cancer from the colon, lungs, kidney, thyroid, prostrate, breasts, or other parts of the body. Therefore, compositions comprising a ROS inhibiting compound or scavenger are useful for treating bone diseases which are secondary to other diseases. In one embodiment, a patient suffering from anorexia or bulimia is administered an effective dose of a ROS inhibiting compound or scavenger to prevent bone loss. In another embodiment, an individual with metastatic cancer of the bone is administered an effective dose of a ROS inhibiting compound or scavenger with or without chemotherapeutic agents to minimize damage to the bone.

The administration of the disclosed compounds can be alone or in combination with other compounds effective at treating various bone disease states. For example, histamine alone can be used to treat a patient suffering from bone loss. Further, the disclosed methods and compounds can be used in combination with standard bone loss treatment regimes, which usually comprise hormone replacement therapy (estrogen, activella®, estratab®, femhrt®, ogen®, ortho-est®, premarin®, premphase®, and prempro® tablets, and climara®, estraderm®, and vivelle® patches), or administration of parathyroid hormone, calcitonins, selective estrogen receptor modulators (SERM), such as tamoxifen, raloxifene and phytoestrogen, calcium, fluoride, vitamin D, vitamin D metabolites, soy isoflavones, and iprifavone. Anti-apoptosis agents, such as transforming growth factor beta (TGF-β), IL-6, estrogen, and bisphosphonates, such as alendronate and risedronate are also contemplated. Also, as discussed above, individuals presenting with metastatic cancer of the bone are administered an effective dose of a ROS inhibiting compound or scavenger along with standard chemotherapy and/or radiation protocols. In the case of menopause-related osteoporosis, a subject can be administered hormone replacement therapy, including the administration of estrogen, concurrently with the administration of a ROS inhibiting compound or scavenger to minimize bone loss or bone cell injury.

The use of the ROS inhibiting or scavenging compounds can be by any of a number of methods well known to those of skill in the art. For oral administration, the ROS inhibiting or scavenging compounds can be incorporated into a tablet, aqueous or oil suspension, dispersible powder or granule, microbead, emulsion, hard or soft capsule, syrup or elixir. The compositions can be prepared according to any method known in the art for the manufacture of pharmaceutically acceptable compositions and such compositions can contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets can be uncoated or can be coated with known techniques to delay disintegration and absorption in the gastrointestinal tract and thereby provide a sustained action over a longer period of time. For example, a time delay material such as glyceryl monostearate or glyceryl stearate alone or with a wax can be employed.

In other embodiments, tablets, capsules or microbeads containing the active ingredient are coated with an enteric coating which prevents dissolution in the acidic environment of the stomach. Instead, this coating dissolves in the small intestine at a more neutral pH. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, D.C., 1998, the entire contents of which are hereby incorporated by reference.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions can contain the ROS inhibiting or scavenging compounds in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules of the compounds, suitable for preparation of an aqueous suspension by the addition of water, provide the active ingredient in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

Administration of the ROS inhibiting or scavenging compounds can also be accomplished via parenteral delivery through subcutaneous, intravenous, intraperitoneal, or intramuscular injection. The compounds can be administered in an aqueous solution with or without a surfactant such as hydroxypropyl cellulose. Dispersions are also contemplated such as those utilizing glycerol, liquid polyethylene glycols, and oils. Injectable preparations can include sterile aqueous solutions or dispersions and powders that can be diluted or suspended in a sterile environment prior to use. Carriers such as solvents or dispersion media contain water, ethanol polyols, vegetable oils and the like can also be added to the disclosed compounds. Coatings such as lecithins and surfactants can be used to maintain the proper fluidity of the composition. Isotonic agents such as sugars or sodium chloride can be added, as well as products intended to delay absorption of the active compounds such as aluminum monostearate and gelatin. Sterile injectable solutions are prepared according to methods well known to those of skill in the art and can be filtered prior to storage and/or use. Sterile powders can be vacuum or freeze dried from a solution or suspension. Sustained or controlled release preparations and formulations can also be used with the disclosed methods. Typically the materials used with the disclosed methods and compositions are pharmaceutically acceptable and substantially non-toxic in the amounts employed.

The disclosed compounds can also be administered by inhalation. In this administration route, histamine, for example, can be dissolved in water or some other pharmaceutically acceptable carrier liquid for inhalation, or provided as a dry powder, and then introduced into a gas or powder that is then inhaled by the patient in an appropriate volume so as to provide that patient with a measured amount of histamine. Examples of the administration of a therapeutic composition via inhalation are described in U.S. Pat. Nos. 6,418,926; 6,387,394; 6,298,847; 6,182,655; 6,132,394; and 6,123,936, which are hereby incorporated by reference.

Infusion devices can be used to deliver the disclosed compounds. Suitable devices include syringe pumps, auto injector systems, implantable pumps, implantable devices, and minipumps. Exemplary devices include the Ambulatory Infusion Pump Drive, Model 30, available from Microject Corp., Salt Lake City, Utah, and the Baxa Syringe Infuser, available from Baxa Corporation, Englewood, Colo. Any device capable of delivering the disclosed compounds in accordance with the methods disclosed herein can be used.

Suitable infusion devices preferably have an effective amount of histamine, histamine agonist, histamine salt, histamine prodrug, NADPH-oxidase inhibitor, histamine dihydrochloride, histamine phosphate, serotonin, a 5HT agonist, a histamine receptor agonist, or a substance which induces the release of an effective therapeutic amount of endogenous histamine, contained therein. The device can be pre-loaded with the desired substance during manufacture, or the device can be filled with the substance just prior to use. Pre-filled infusion pumps and syringe pumps are well known to those of skill in the art. The active substance can be part of a formulation which includes a controlled release carrier, if desired. A controller is used with the device to control the rate of administration and the amount of substance to be administered. The controller can be integral with the device or it can be a separate entity. It can be pre-set during manufacture, or set by the user just prior to use. Such controllers and their use with infusion devices are well known to those of skill in the art.

Controlled release vehicles are well known to those of skill in the pharmaceutical sciences. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released are also well known and can be used in the disclosed methods.

In one embodiment, the disclosed compounds are administered through a topical delivery system. The controlled release components described above can be used as the means to deliver the disclosed compounds. A suitable topical delivery system comprises the disclosed compounds in concentrations taught herein, a solvent, an emulsifier, a pharmaceutically acceptable carrier material, penetration enhancing compounds, and preservatives. Examples of topically applied compositions include U.S. Pat. Nos. 5,716,610 and 5,804,203, which are hereby incorporated by reference. The compositions can further include components adapted to improve the stability or effectiveness of the applied formulation, such as preservatives, antioxidants, skin penetration enhancers and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

In another embodiment, the disclosed compounds can be administered directly to the bone using biocompatible and/or bioresorbable matrices of natural or synthetic origin. Preformed, implantable and injectable polymeric formulations can be used. The ROS inhibiting or scavenging compounds can be incorporated into the matrices such that controlled release or sustained delivery of the compounds is achieved. Examples of biocompatible and bioresorbable matrices are known in the art and include, but are not limited to, porous biodegradable polymers, biodegradable hydrogels, hybridized polymers, biodegradable polymer films, polyethylene glycol copolyesters, polymer sealants, porous biodegradable scaffolds, putty-like biodegradable scaffolds, and demineralized bone matrices.

Controlled release preparations can be achieved by the use of polymers to complex or absorb the ROS inhibiting or scavenging compound. The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the ROS inhibiting or scavenging compound is dissolved in an aqueous constituent to gradually release over time, can be prepared by copolymerization of hydrophilic mono-olefinic monomers such as ethylene glycol methacrylate. Matrix devices, wherein the ROS inhibiting or scavenging compound is dispersed in a matrix of carrier material, can be used. The carrier can be porous, non-porous, solid, semi-solid, permeable or impermeable. Alternatively, a device comprising a central reservoir of the ROS inhibiting or scavenging compound surrounded by a rate controlling membrane can be used to control the release of the ROS inhibiting or scavenging compound. Rate controlling membranes include ethylene-vinyl acetate copolymer or butylene terephthalate/polytetramethylene ether terephthalate. Use of silicon rubber depots are also contemplated.

Controlled release oral formulations are also well known. In one embodiment, the active compound is incorporated into a soluble or erodible matrix, such as a pill or a lozenge. Such formulations are well known in the art. An example of a lozenge used to administer pharmaceutically active compounds is U.S. Pat. No. 5,662,920, which is hereby incorporated by reference. In another example, the oral formulations can be a liquid used for sublingual administration. An example of pharmaceutical compositions for liquid sublingual administration of the disclosed compounds are taught in U.S. Pat. No. 5,284,657, which is hereby incorporated by reference. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

For the purpose of parenteral administration, ROS inhibiting or scavenging compounds can be combined with distilled water, preferably buffered to an appropriate pH and having appropriate (e.g., isotonic) salt concentrations. The compounds can also be provided as a liquid or as a powder that is reconstituted before use. They can be provided as prepackaged vials, syringes, or injector systems.

The disclosed compounds, such as histamine, can also be provided in septum-sealed vials in volumes ranging from about 0.5 to about 100 ml for administration to an individual. The vials are preferably sterile. The vials can optionally contain an isotonic carrier medium and/or a preservative. Any desired amount of histamine or other ROS inhibitory compound can be used to give a desired final concentration. In a preferred embodiment, the ROS inhibiting or scavenging concentration is between about 0.01 mg/ml and about 100 mg/ml. More preferably, the ROS inhibiting or scavenging compound concentration is between about 0.1 and about 50 mg/ml. Most preferably, the ROS inhibiting or scavenging compound concentration is between about 1 mg/ml and about 10 mg/ml. At the lower end of the volume range, it is preferred that individual doses are administered, while at the higher end it is preferred that multiple doses are administered.

In another embodiment, transdermal patches, steady state reservoirs sandwiched between an impervious backing and a membrane face, and transdermal formulations, can also be used to deliver ROS inhibiting or scavenging compounds. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference. In one embodiment, the steady state reservoir carries doses of histamine or other ROS production and release inhibitory or scavenging compounds in doses from about 0.2 to about 200 mg per day.

Present transdermal patch systems are designed to deliver smaller doses over longer periods of time, up to days and weeks. A preferred delivery system for the disclosed compounds would specifically deliver an effective dose of, for example, histamine, in a range of between about 2 and about 60 minutes, depending upon the dose, with a preferred dose being delivered within about 20 to 30 minutes. These patches allow rapid and controlled delivery of a compound which inhibits or scavenges ROS. A rate-controlling outer microporous membrane, or micropockets of the disclosed compounds dispersed throughout a silicone polymer matrix, can be used to control the release rate. Such rate-controlling means are described in U.S. Pat. No. 5,676,969, which is hereby incorporated by reference. In another embodiment, the histamine or other ROS inhibiting or scavenging compound is released from the patch into the skin of the patient in about 20 to 30 minutes or less. In one embodiment, the compound is released from the patch at a rate of between about 0.025 mg to about 6 mg per minute for a dose of between about 0.2 mg and about 200 mg per patch.

These transdermal patches and formulations can be used with or without use of a penetration enhancer such as dimethylsulfoxide (DMSO), combinations of sucrose fatty acid esters with a sulfoxide or phosphoric oxide, or eugenol. The use of electrolytic transdermal patches is also within the scope of the methods disclosed herein. Electrolytic transdermal patches are described in U.S. Pat. Nos. 5,474,527, 5,336,168, and 5,328,454, the entire contents of which are hereby incorporated by reference.

In another embodiment, transmucosal patches can be used to administer the disclosed compounds. An example of such a patch is found in U.S. Pat. No. 5,122,127, which is hereby incorporated by reference. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface. Accordingly, drug delivery rates can be adjusted by altering the size of the contact area.

The housing is preferably constructed of a material which is nontoxic, chemically stable, and non-reactive with the disclosed compounds. Possible construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the disclosed compounds delivery a suitable dose of those compounds over a predetermined period of time. Compositions and methods of manufacturing compositions capable of absorption through the mucosal tissues are taught in U.S. Pat. No. 5,288,497, which is hereby incorporated by reference. One of skill in the art could readily include the disclosed compounds and related compositions.

The steady state reservoirs for use with the disclosed compounds are composed of compounds known in the art to control the rate of drug release. In one embodiment, the transmucosal patch delivers a dose of a ROS inhibiting or scavenging compound over a period of time from about 2 to about 60 minutes. The steady state reservoir contained within the housing carries doses of histamine or other ROS production and release inhibitory compounds in doses from about 0.1 to about 200 mg per patch. Transdermal patches that can be worn for several days and that release the disclosed compounds over that period of time are also contemplated. The reservoirs can also contain permeation or penetration enhancers, as discussed above, to improve the permeability of the disclosed compounds across the mucosal tissue.

Another method to control the release of the disclosed compounds is to incorporate the ROS inhibiting or scavenging compound into particles of a polymeric material such as polyesters, polyamino acids, hydrogels, poly lactic acid, or ethylene vinylacetate copolymers.

Alternatively, instead of incorporating the ROS inhibiting or scavenging compounds into these polymeric particles, the disclosed compounds can be entrapped in microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions. Such technology is well known to those of ordinary skill in pharmaceutical sciences.

Preferably, the compounds that inhibit ROS are injected, infused, or released into the patient at a rate of from about 0.025 to about 10 mg/min. A rate of about 0.1 mg/min is preferred. The disclosed compounds are preferably administered over a period of time ranging from about 1 to about 30 minutes, with an upper limit of about 20 minutes being preferred, such that the total daily adult dose of ROS inhibiting or scavenging compound ranges from between about 0.1 to about 200 mg, with about 0.2 to about 100 mg being preferred.

In another embodiment, a ROS inhibiting or scavenging compound at approximately 0.2 to about 200 mg, or about 3 to about 2500 μg/kg body weight, in a pharmaceutically acceptable form can be administered. ROS scavenging compounds can also be administered in combination with the ROS production and release inhibitory compounds described above.

The treatment can also include periodically boosting patient blood ROS inhibiting or scavenging compound levels by administering additional compound in amounts ranging from about 0.2 to about 200 mg, or about 3 to about 2500 μg/kg body weight, one to four times per day over a period of one to two weeks at regular intervals, such as daily, bi-weekly, or weekly in order to establish blood levels of ROS inhibiting or scavenging compound at a beneficial concentration such that ROS production and release is inhibited. The administration can be by any of the means described above. The treatment is continued until the causes of the patient's underlying disease state is controlled or eliminated.

Administration of each dose of ROS inhibiting or scavenging compound can occur from once a day to up to about four times a day, with twice a day being preferred. Administration can be subcutaneous, intraperitoneal, intravenous, intramuscular, intraocular, oral, transdermal, intranasal, or rectal and can utilize direct hypodermic or other injection or infusion means, or can be mediated by a controlled release mechanism of the type disclosed above. Any controlled release vehicle or infusion device capable of administering a therapeutically effective amount of the disclosed compounds over a period of time ranging from about 1 to about 30 minutes can be used. In one embodiment, intranasal delivery is accomplished by using a solution of ROS inhibiting or scavenging compound in an atomizer or nebulizer to produce a fine mist which is introduced into the nostrils. For rectal delivery, ROS inhibiting or scavenging compound is formulated into a suppository using methods well known in the art.

In another embodiment, the ROS inhibiting or scavenging compound can be administered orally. When administered orally, the compound can be administered in capsule, tablet, granule, spray, syrup, or other such form. In one embodiment, the composition can be formulated as a tablet comprising between about 10 mg to about 2 grams of active ingredient. For example, such a tablet can include 10, 20, 50, 100, 200, 500, 1,000, or 2,000 milligrams of ROS inhibiting or scavenging compound. Preferably, the amount of ROS inhibiting or scavenging compound in a tablet is about 100 mg. In some embodiments, the composition includes histamine protectors such as diamine oxidase inhibitors, monoamine oxidase inhibitors and n-methyl transferases.

Compounds that scavenge ROS can be administered in an amount of from about 0.2 to about 200 mg/day; more preferably, the amount is from about 0.5 to about 20 mg/day; and even more preferably, the amount is from about 1 to about 5 mg/day. In each case, the dose depends on the activity of the administered compound. The foregoing doses are appropriate for the enzymes listed above that include catalase, superoxide dismutase (SOD), glutathione peroxidase and ascorbate peroxidase. Appropriate doses for any particular host can be readily determined by empirical techniques well known to those of ordinary skill in the art.

Non-enzymatic ROS scavengers can be administered in amounts empirically determined by one of ordinary skill in the art. For example, vitamins A and E can be administered in doses from about 1 to about 5000 IU per day. Vitamin C can be administered in doses from about 1 μg to about 10 gm per day. Minerals such as selenium and manganese can be administered in amounts from about 1 picogram to about 1 milligram per day. These compounds can also be administered as a protective or preventive treatment for ROS-mediated disease states.

As noted above, in addition to histamine, histamine dihydrochloride, histamine phosphate, other histamine salts, histamine esters, histamine congeners, histamine prodrugs, and H₂ receptor agonists, the use of serotonin, 5HT agonists, and compounds which induce release of histamine from the patient's own tissues are all included within the disclosed compounds and methods. Retinoic acid, other retinoids such as 9-cis-retinoic acid and all-trans-retinoic acid, IL-3 and ingestible allergens are compounds that are known to induce the release of endogenous histamine. These compounds can be administered to the patient by the means described above, including oral, intravenous, intramuscular, subcutaneous, and other approved routes. The rate of administration preferably results in a release of endogenous histamine resulting in a blood plasma level of histamine of about 20 nmol/dl.

Administration of each dose of a compound which induces histamine release can occur from once per day to up to about four times a day, with twice per day being preferred. Administration can be subcutaneous, intravenous, intramuscular, intraocular, oral, or transdermal, and can incorporate a controlled release mechanism of the type disclosed above. Any controlled release vehicle capable of administering a therapeutically effective amount of a compound which induces histamine release over a period of time ranging from about one to about thirty minutes can be used. Additionally, the compounds, compositions, and formulations of embodiments of the invention can be administered as needed to ease the pain or discomfort of the subject.

The following examples teach various methods for treating bone disease with the disclosed ROS production and release inhibiting compounds. These examples are illustrative only and are not intended to limit the scope of the claims. The treatment methods described below can be optimized using empirical techniques well known to those of ordinary skill in the art. Moreover, artisans of ordinary skill would be able to use the teachings described in the following examples to practice the full scope of the claims. Although it is stated in the examples that the administration of a ROS inhibiting or scavenging compound can be given in a single dose, it is obvious that the compounds can be distributed over longer periods of time. Moreover, the daily dose can be administered as a single dose or it can be divided into several doses.

### EXAMPLES

### Example 1

### Inhibition of Bone Resorption

Subjects suffering from bone loss exacerbated by osteoclast-produced ROS are identified. The subjects are separated into 11 groups of 10 subjects each. Subjects in Groups 1 through 10 are administered an effective dose of histamine, histamine agonists, histamine salts, histamine prodrugs, NADPH-oxidase inhibitors, histamine dihydrochloride, histamine phosphate, serotonin, 5HT agonists, or histamine receptor agonists, respectively. Subjects in Group 11 are administered a placebo. The rate of bone loss is reduced and the rate of bone healing is accelerated for subjects in Groups 1 through 10 as compared to subjects in Group 11.

### Example 2

### Treatment of Bone Diseases

Individuals suffering from bone diseases, such as osteoporosis, metastatic cancers of the bone, periodontal disease, osteopenia, osteomalacia, osteolytic bone disease, multiple myeloma, osteoclerotic lesions, osteoblastic lesions, fractures, osteoarthritis, infective arthritis, ankylosing spondylitis, gout, fibrous dyplasia, and Paget's disease of the bone, are identified. The individuals are divided into 5 groups of 25 individuals each. Individuals in Groups 1 through 4 are intravenously administered 0.5 mg, 1 mg, 5 mg, and 20 mg of histamine prodrugs, respectively. Individuals in Group 5 are administered a placebo. The histamine prodrugs or placebos are administred in conjunction with standard bone loss treatment regimes, such as hormone replacement therapy, parathyroid hormone, calcitonins, selective estrogen receptor modulators (SERM), calcium, fluoride, vitamin D, vitamin D metabolites, soy isoflavones, and iprifavone, transforming growth factor beta (TGF-β), IL-6, estrogen, and bisphosphonates, such as alendronate and risedronate. The rate of bone healing for Groups 1 through 4 is accelerated relative to the rate of bone healing for the placebo group. In addition, the rate of bone healing for Groups 1 through 4 is accelerated in a dose responsive manner.

### Example 3

### Treatment of Osteoporosis

Individual suffering from various types of osteoporosis, including type I and type II osteoporosis, age-related osteoporosis, disuse osteoporosis, diabetes-related osteoporosis, and steroid-related osteoporosis, are identified. The individuals are separated into 5 groups of 20 individuals. Individuals in Groups 1 through 4 are orally administered 50 mg, 100 mg, 200 mg, and 1,000 mg of histamine, respectively. Individuals in Group 1 are administered a placebo. The rate of bone loss is reduced for Groups 1 through 4 relative to the rate of bone loss for the placebo group.

### Example 4

### Treatment of Metastatic Cancers of the Bone

Individuals suffering from metastatic cancers of the spine, pelvis, limbs, hip, and skull are identified. The individuals are intramuscularly administered 10 mg of NADPH-oxidase inhibitors or a placebo. The rate of bone loss is minimized and the rate of bone healing is accelerated for individuals who received the NADPH-oxidase inhibitor as compared to individuals who received a placebo.

### Example 5

### Inhibition of Bone Loss Associated with Primary and Secondary Hyperparathyroidism

Individuals suffering from bone loss associated with primary and secondary hyperparathyroidism are identified. The individuals are orally administered 125 mg of histamine or a placebo. The rate of bone loss is reduced and the trauma to bone cells is minimized in individuals who received histamine.

### Example 6

### Treatment of Osteomyelitis

Individuals suffering from osteomyelitis are identified. The individuals are orally administered 75 mg of histamine phosphate in conjunction with antibiotics or antibiotics alone. Bone healing is accelereated in individuals who received histamine.

### Example 7

### Treatment of Bone Loss Associated with Cancer Treatments

Individuals suffering from cancer and being treated with hormone therapy are identified. The individuals are divided into 11 groups of 10 each. In conjunction with hormone therapy, Groups 1 through 10 are orally administered 100 mg of histamine, histamine agonists, histamine salts, histamine prodrugs, NADPH-oxidase inhibitors, histamine dihydrochloride, histamine phosphate, serotonin, 5HT agonists, or histamine receptor agonists, respectively. Group 11 receives a placebo in conjunction with hormone therapy. Bone loss is inhibited and bone healing is accelerated in Groups 1 through 10 as compared to the placebo group.

### Example 8

### Treatment of Bone Loss Associated with Steroid Therapy

Individuals suffering from bone loss associated with steroid therapy, including treatment with corticosteroids, such as betamethasone, budesonide, cortisone dexamethasone, hydrocortisone, methylprednisolone, prednisolone, prednisone, and triamcinolone, are identified. In conjunction with steroid therapy, the individuals are intravenously administered 5 mg of a ROS scavenger or a placebo. Individuals receiving histamine dihydrochloride in conjunction with steroid therapy exhibit reduced bone loss as compared to individuals receiving steroid therapy alone.

### Example 9

### Treatment of Bone Loss Associated with Thyroid Treatments

Individuals suffering from thyroid conditions and being treated with thyroid hormones, such as thyroxine, are identified. The individuals are separated into 5 groups of 20 individuals. Individuals in Groups 1 through 4 are orally administered 50 mg, 100 mg, 200 mg, and 1,000 mg of a histamine agonist, respectively. Individuals in Group 1 are administered a placebo. The rate of bone loss is reduced for Groups 1 through 4 relative to the rate of bone loss for the placebo group. Bone loss is inhibited in a dose responsive manner.

### Example 10

### Treatment of Bone Loss Associated with Anticonvulsants

Individuals suffering from bone loss associated with anticonvulsants, such as barbituates, phenoarbital, phenyloin, and benzodiazepines, are identified. The individuals are intravenously administered 1 mg of a histamine receptor agonist or a placebo in conjunction with the anticonvulsants. Bone loss is inhibited and wound healing accelerated in individuals receiving the histamine receptor agonist as compared to the placebo group.

The foregoing description details certain embodiments of the invention. It will be appreciated, however, that no matter how detailed the foregoing appears in text, the invention can be practiced in many ways. As is also stated above, it should be noted that the use of particular terminology when describing certain features or embodiments of the invention should not be taken to imply that the terminology is being re-defined herein to be restricted to including any specific characteristics of the features or embodiments of the invention with which that terminology is associated. The scope of the invention should therefore be construed in accordance with the appended claims and any equivalents thereof.

1. A method for treating or preventing reactive oxygen species (ROS)-mediated oxidative damage to bone cells and tissues of a subject comprising:
identifying an individual suffering from or at risk for a bone disease caused or exacerbated by ROS-mediated oxidative damage; and
administering to said individual a compound effective to reduce the amount of ROS.

2. The method of claim 1, wherein said ROS-mediated oxidative damage is enzymatically produced ROS-mediated oxidative damage.

3. The method of claim 1, wherein said ROS-mediated oxidative damage is cellular-derived ROS-mediated oxidative damage

4. The method of claim 3, wherein said cellular-derived ROS-mediated oxidative damage is osteoclast-derived ROS-mediated oxidative damage.

5. The method of claim 1, wherein said bone disease is selected from the group consisting osteoporosis, periodontal disease, osteopenia, osteomalacia, osteolytic bone disease, primary and secondary hyperparathyroidism, multiple myeloma, metastatic cancers of the bone, for example, of the spine, pelvis, limbs, hip, and skull, osteomyelitis, osteoclerotic lesions, osteoblastic lesions, fractures, osteoarthritis, infective arthritis, ankylosing spondylitis, gout, fibrous dyplasia, and Paget's disease of the bone.

6. The method of claim 5, wherein said osteoporosis is selected from the group consisting of type I osteoporosis, type II osteoporosis, age-related osteoporosis, disuse osteoporosis, diabetes-related osteoporosis, and steroid-related osteoporosis.

7. The method of claim 1, wherein said compound is selected from the group consisting of a compound that inhibits the production or release of cellular-derived and enzymatically produced ROS, a ROS scavenger, and combinations thereof.

8. The method of claim 7, wherein said compound effective to inhibit the production or release of ROS is selected from the group consisting of histamine, histamine receptor agonists, histamine salts, histamine prodrugs, NADPH-oxidase inhibitors, serotonin, serotonin (5HT) receptor agonists, and substances which induce the release of an effective therapeutic amount of endogenous histamine.

9. The method of claim 7, wherein the administration of the ROS scavenger results in ROS scavenger catalyzed decomposition of ROS.

10. The method of claim 7, wherein said ROS scavenger is selected from the group consisting of catalase, superoxide dismutase, glutathione peroxidase, and ascorbate peroxidase.

11. The method of claim 7, wherein said ROS scavenger is selected from the group consisting of vitamin A, vitamin E, and vitamin C.

12. The method of claim 1, wherein said compound is administered in multiple doses.

13. The method of claim 1, wherein the administration of the compound is accomplished by a method selected from the group consisting of injection, intramuscular injection, intravenous injection, implantation infusion device, inhalation, and transdermal diffusion.

14. The method of claim 1, wherein said compound is administered in a dosage of about 0.2 mg to about 200 mg.

15. The method of claim 1, wherein said compound is administered orally.

16. The method of claim 15, wherein said compound is in a form selected from the group consisting of capsules, tablets, granules, sprays, and syrups.

17. The method of claim 1, wherein bone healing is accelerated.

18. A method for accelerating bone healing comprising:
administering to a subject in need thereof an amount of a compound effective to reduce the amount of ROS.

19. The method of claim 18, wherein said compound is selected from the group consisting of a compound that inhibits the production or release of ROS, a ROS scavenger, and combinations thereof.

20. The method of claim 19, wherein said compound effective to inhibit the production or release of cellular-derived and enzymatically produced ROS is selected from the group consisting of histamine, histamine receptor agonists, histamine salts, histamine prodrugs, NADPH-oxidase inhibitors, serotonin, serotonin (5HT) receptor agonists, and substances which induce the release of an effective therapeutic amount of endogenous histamine.

21. The method of claim 19, wherein the scavenger is selected from the group consisting of catalase, superoxide dismutase, glutathione peroxidase, and ascorbate peroxidase.

22. The method of claim 18, wherein the compound is administered in a dosage of about 0.2 mg to about 200 mg.
